(19) United States
(12) Patent Application Publication
(10) Pub. No.: US 2024/0350086 A1
(43) Pub. Date: Oct. 24, 2024

(54) ADHESION DETECTION FOR A MEDICAL PATCH

(71) Applicant: Given Imaging LTD., Yoqneam (IL)

(72) Inventors: Eyal David, Atlit (IL); Iddo Diukman, Zihron Yaakov (IL); Baruch Gruman, Lapid (IL)

(21) Appl. No.: 18/763,010

(22) Filed: Jul. 3, 2024

Related U.S. Application Data
(63) Continuation-in-part of application No. 17/917,021, filed on Oct. 5, 2022.
(60) Provisional application No. 63/028,838, filed on May 22, 2020.

(51) Int. Cl. A61B 5/00

(52) U.S. Cl. CPC A61B 5/6843; A61B 5/6833; A61B 5/74

(57) ABSTRACT

A patch includes: an adhesive layer configured to adhere to skin of a person, and a communication circuit that includes a current sensor and a resonant circuit. The resonant circuit includes a coil antenna and a capacitor. The current sensor is configured to sense current in the communication circuit over time, where the sensed current has a known relationship to current through the coil antenna. The patch further includes at least one of a circuit or a processor configured to at least perform: determining that a change in the current through the coil antenna is greater than a threshold change, and providing, based on the determination, an indication that the adhesive layer is at least partially detached from the skin of the person.

### CROSS-REFERENCE TO RELATED APPLICATIONS

The present application is a continuation-in-part of U.S. patent application Ser. No. 17/917,021, filed Oct. 5, 2022, which is a U.S. National Stage Application filed under 35 U.S.C. § 371 (a) of International Patent Application No. PCT/IL2021/050582, filed May 20, 2021, which claims the benefit of and priority to U.S. Provisional Application No. 63/028,838, filed May 22, 2020. All of the foregoing applications are hereby incorporated by reference herein in their entirety.

### TECHNICAL FIELD

The present disclosure is in the field of medical patches, in particular, medical patches configured for communicating with in-vivo devices.

### BACKGROUND

Medical patches (also referred as skin patches) are devices configured for being fitted to a patient's skin for one of two main purposes: medicative and monitoring. Medicative patches, commonly referred to as transdermal patches, comprise a medication and are configured for providing this medication to the patient via the skin, either by puncturing the skin (with a needle) or by transdermal diffusion. Monitoring patches, one the other hand, are configured for sensing different parameters of the patient (e.g. micro-movements, electricity, pulse etc.) and for communicating with in-vivo devices as very common with pacemakers.

Medical patches may be applied to the skin in various ways, one way being an adhesive layer.

Acknowledgement of the above references herein is not to be inferred as meaning that these are in any way relevant to the patentability of the presently disclosed subject matter.

### SUMMARY

In accordance with aspects of the present disclosure, a patch includes: an adhesive layer configured to adhere to skin of a person, and a communication circuit that includes a current sensor and a resonant circuit. The resonant circuit includes a coil antenna and a capacitor. The current sensor is configured to sense current in the communication circuit over time, where the sensed current has a known relationship to current through the coil antenna. The patch further includes at least one of a circuit or a processor configured to at least perform: determining that a change in the current through the coil antenna is greater than a threshold change, and providing, based on the determination, an indication that the adhesive layer is at least partially detached from the skin of the person.

In various embodiments of the patch, the communication circuit operates at a working frequency, where the working frequency is configured to be a resonant frequency of the resonant circuit when the adhesive layer is fully adhered to the skin of the person.

In various embodiments of the patch, the working frequency is not the resonant frequency of the resonant circuit when the adhesive layer is at least partially detached from the skin of the person.

In various embodiments of the patch, the determining that the change in the current through the coil antenna is greater than a threshold change includes determining that an increase in the current through the coil antenna is greater than the threshold change.

In various embodiments of the patch, the determining that the change in the current through the coil antenna is greater than a threshold change includes determining that a decrease in the current through the coil antenna is greater than the threshold change.

In various embodiments of the patch, the communication circuit is configured to transmit signals to an in-vivo device within the person, where the change in the current through the coil antenna is sensed during a transmission by the communication circuit.

In accordance with aspects of the present disclosure, a method is disclosed in a patch that includes an adhesive layer configured to adhere to skin of a person, and a communication circuit that includes a current sensor and a resonant circuit, where the resonant circuit includes a coil antenna and a capacitor. The method includes: sensing, by the current sensor, current in the communication circuit over time, where the sensed current has a known relationship to current through the coil antenna; determining that a change in the current through the coil antenna is greater than a threshold change; and providing, based on the determination, an indication that the adhesive layer is at least partially detached from the skin of the person.

In various embodiments of the method, the method further includes: operating the communication circuit at a working frequency, where the working frequency is configured to be a resonant frequency of the resonant circuit when the adhesive layer is fully adhered to the skin of the person.

In various embodiments of the method, the working frequency is not the resonant frequency of the resonant circuit when the adhesive layer is at least partially detached from the skin of the person.

In various embodiments of the method, the determining that the change in the current through the coil antenna is greater than a threshold change includes determining that an increase in the current through the coil antenna is greater than the threshold change.

In various embodiments of the method, the determining that the change in the current through the coil antenna is greater than a threshold change includes determining that a decrease in the current through the coil antenna is greater than the threshold change.

In various embodiments of the method, the method further includes: transmitting, by the communication circuit, signals to an in-vivo device within the person, where the change in the current through the coil antenna is sensed during a transmission by the communication circuit.

In accordance with aspects of the present disclosure, a non-transitory processor readable medium stores instructions which are executed by at least one processor of a patch. The patch includes an adhesive layer configured to adhere to skin of a person, and a communication circuit that includes a current sensor, and a resonant circuit. The resonant circuit includes a coil antenna and a capacitor. The instructions, when executed by at least one processor of the patch, causes the patch at least to perform: accessing data representing sensed current, sensed by the current sensor, of current in the communication circuit over time, where the sensed current has a known relationship to current through the coil antenna; determining that a change in the current through the coil antenna is greater than a threshold change; and providing, based on the determination, an indication that the adhesive layer is at least partially detached from the skin of the person.

In various embodiments of the non-transitory processor readable medium, the instructions, when executed by the at least one processor, further cause the patch to at least perform: operating the communication circuit at a working frequency, where the working frequency is configured to be a resonant frequency of the resonant circuit when the adhesive layer is fully adhered to the skin of the person.

In various embodiments of the non-transitory processor readable medium, the working frequency is not the resonant frequency of the resonant circuit when the adhesive layer is at least partially detached from the skin of the person.

In various embodiments of the non-transitory processor readable medium, the determining that the change in the current through the coil antenna is greater than a threshold change includes determining that an increase in the current through the coil antenna is greater than the threshold change.

In various embodiments of the non-transitory processor readable medium, the determining that the change in the current through the coil antenna is greater than a threshold change includes determining that a decrease in the current through the coil antenna is greater than the threshold change.

In various embodiments of the non-transitory processor readable medium, the instructions, when executed by the at least one processor, when further cause the patch at least to perform: transmitting, by the communication circuit, signals to an in-vivo device within the person, where the change in the current through the coil antenna is sensed during a transmission by the communication circuit.

In accordance with one aspect of the subject matter of the present application, there is provided a patch configured for being applied to a patient's skin, said patch comprising a contact area configured for being in direct contact with the patient's skin when applied thereto, and a detector configured for detecting contact between the contact area and the patient's skin.

The patch may further comprise an indicator module associated with the detector and configured, based on input therefrom, to indicate states of contact of the contact area with the patient's skin. In particular, the detector may provide at least one of the following:
a positive contact indication signal configured indicating that the contact area is in adequate contact with the patient's skin; and
a negative contact indication signal configured for indicating that the contact area is not in adequate contact with the patient's skin.

In addition to the above, the indicator module may comprise several indication signals, each relating to a different state of contact between the contact area and the patient's skin. In accordance with a specific example, the contact area may comprise two or more different regions and the indicator may be configured for providing, for at least some of each of these regions a positive/negative indication signal.

The patch of the present application can thus alert the patient or a medical practitioner monitoring the patient regarding a malfunction. In case of the such malfunction, the patient or medical practitioner may either reattach the patch such that the contact area is properly fitted to the patient's skin or replace the patch.

The detector may be based on any one of the following mechanisms, but not limited thereto: electric current, electric capacitance, electric induction, heat capacitance and chemical reaction.

In accordance with a specific implementation, the patch may comprise a communication module configured for providing communication between the patch and an in-vivo device located within the patient. The communication module may be further configured for providing communication with one or more ex-vivo devices. The communication module may comprise a power source and an antenna arrangement configured for providing the above desired communication.

In accordance with a specific example, the in-vivo device may be a swallowable endoscopic capsule configured for providing data regarding the patient's GI. Under this example, the communication patch may be configured for staying in communication with a movable in-vivo device traversing the patient's GI. As such, detachment of the patch from the patient's skin may impede communication between the patch and the capsule, which may, under extreme circumstances, make the entire endoscopic procedure useless. In addition, noting that such a procedure (or indeed any endoscopic/colonoscopic procedure) requires a substantial preparation of the patient (laxatives, bowel cleaning etc.) which is usually unpleasant, providing a direct indication that something is wrong may spell the difference between proper completion of the procedure and going through the preparation a second time.

The patch may comprise a first layer with a rear face constituting the contact area, and a front face facing away from the patient. In accordance with one example, the first layer may have embedded therein the communication module and additional patch components. In accordance with another example, the patch may comprise additional layers configured for accommodating the communication module and any additional patch components.

Under a specific arrangement, the patch is designed such that none of the electrical components of the patch are in direct contact with the patient's skin. Under this arrangement, for example, the detector may be a capacitance detector. The capacitance detector is configured for measuring the electrical capacitance and providing said reading to the indicator module or to a processor which is, in turn, associated with the indicator module.

The capacitance detector may be calibrated to have a baseline reading corresponding to a state in which the contact area is fully detached from the patient's skin. Thus, when the patch is properly adhered to the patient's skin, the capacitance detector will detect a spike in capacitance compared to the baseline reading, and when a portion of the contact area becomes detached from the patient's skin, the capacitance detector will detect a drop in capacitance.

It has been discovered that detachment of even a portion of the contact area from the patient's skin can drastically affect the capacitance measured by the capacitance detector, which allows providing an accurate monitoring mechanism for proper attachment of the patch to the patient's skin.

It should be appreciated that the distance at which degradation of the sensitivity of the capacitance detector depends on various parameters, including, but not limited to the initial distance for which the baseline sensitivity is calibrated to, size of the sensor and the SNR. In accordance with a particular example of the present application, the sensor size and SNR may be calibrated such that any increase of the initial distance by more than at least 30% will yield a significant change in capacitance, allowing its detection. As a specific example, the initial distance of the sensor from the skin may range between 1.5-5 mm, more particularly between 2-4 mm, and even more particularly around 3 mm.

The baseline can be chosen, for example, as the baseline capacitance when the patch is fully adhered to the patient's skin. Alternatively, the baseline may also be chosen as the baseline capacitance when the patch is completely detached from the patient's skin (e.g. surrounded by air).

The capacitance detector may be connected to the antenna arrangement of the communication module and utilize components of the antenna arrangement as part of the detector. The capacitance detector may operate under the following scheme:

In general, the scheme may support more than one capacitive sensor. Each capacitive sensor has a sensing electrode connected to an oscillator, and a reference electrode connected to the ground plane of the circuit. The shape and the distance between electrodes may vary and depend on use case and sensitivity optimization.

Each oscillator, when enabled, generates a square wave signal at its output. The frequency of the square wave may vary in a certain range, inverse-proportional to the sensing capacitor value. The oscillator output signal, chosen by the selector, is used as a counter clock. Before each measurement the counter is reset and then enabled for a constant time window. At the end of the window the counter readout is proportional to the oscillator frequency and inverse-proportional to the sensor capacitance. The circuit is calibrated with 2 known capacitors, so the offset and the slope constants are recorded in NVM (Non Volatile Memory). Using these constants and the counter readout, the CPU calculates the real capacitance measured by the sensor.

Alternatively, the capacitance detection may also be performed by Self capacitance, which is relative to earth ground. In order to measure the self capacitance, charge is transferred between three difference capacitors. First, the charge stored on a Vreg capacitor (recommended value of 1 uF), is used to charge an external unknown capacitance during the charge phase. Second, the charge from the external capacitance is transferred to an internal sampling capacitor. During this transfer phase when charge is moved from the external capacitor to the sample capacitor the Vreg capacitor is refilled with charge by an LDO. These charge and transfer phases are repeated until the voltage on the internal sampling capacitor changes by the desired amount. This voltage can be changed to allow for a wide range of external capacitances.

The contact area may comprise an adhesive layer configured for allowing fitting of the patch to the patient's skin. In accordance with a specific example of the subject matter of the present application, the adhesive material may be chosen such that it provides, on the one hand, the required adhesion between the patch and the patient's skin, and, on the other hand, the required dielectric properties allowing the capacitance detector to properly distinguish between different adhesion states of the patch. Examples of the adhesive material which may be used may include, but are not limited to:

The capacitive sensor may reside on the inner side of the foam layer, closer to the patient's skin and separated therefrom by the adhesive layer alone, or, alternatively, on the outer side of the foam layer, distanced from the patient's skin, or any other position therebetween.

In accordance with another design example, the detection of adhesion to the patient's skin may be performed based on load resistance and a resonance capacitor. Specifically, the patch may comprise an antenna coil and a resonance capacitor at working frequency. Under this configuration, the load to the antenna driver is pure active resistance composed mainly by losses caused by human tissue attachment of the antenna coil.

Detaching the patch from the body decreases the human tissue loss, and therefore decreases the load resistance of the drive amplifier. This resistance change may be used to monitor the attachment of the patch to the body.

The details of one or more embodiments of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic front view of a patch in accordance with and embodiment of the present application, when applied to a patient's abdominal region;

FIG. 2A is a schematic front view of the patch shown in FIG. 1;

FIG. 2B is a schematic exploded view of the patch shown in FIG. 2A;

FIG. 3 is a schematic diagram of a capacitance detector implemented in the patch shown in FIGS. 1 to 2B;

FIG. 4; is a schematic graph showing readings taken by the capacitance detector when attached and detached from the body;

FIG. 5 is a schematic diagram of another example of a capacitance detector which can be used in the patch shown in FIGS. 1 to 2B;

FIG. 6 is a schematic diagram of an example of a an adhesion detector based on a communication circuit, which can be used in the patch shown in FIGS. 1 to 2B;

FIG. 7 is a diagram of an equivalent circuit for the adhesion detector of FIG. 6, in accordance with aspects of the present disclosure;

FIG. 8 is a diagram of eddy currents generated when the patch is fully adhered to a person, in accordance with aspects of the present disclosure;

FIG. 9 is a graph of an increase in current in the communication circuit of FIG. 8, in accordance with aspects of the present disclosure;

FIG. 10 is a graph of a decrease in current in the communication circuit of FIG. 8, in accordance with aspects of the present disclosure;

FIG. 11 is a graph of an effect that increases current and an effect that decreases current in the communication circuit of FIG. 8, in accordance with aspects of the present disclosure; and

FIG. 12 is a flow diagram of an operation for adhesion detection, in accordance with aspects of the present disclosure.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF EMBODIMENTS

In the following description, certain specific details are set forth in order to provide a thorough understanding of disclosed aspects. However, one skilled in the relevant art will recognize that aspects may be practiced without one or more of these specific details or with other methods, components, materials, etc. In other instances, well-known structures associated with transmitters, receivers, or transceivers have not been shown or described in detail to avoid unnecessarily obscuring descriptions of the aspects.

Reference throughout this specification to “one aspect” or “an aspect” means that a particular feature, structure, or characteristic described in connection with the aspect is included in at least one aspect. Thus, the appearances of the phrases “in one aspect” or “in an aspect” in various places throughout this specification are not necessarily all referring to the same aspect. Furthermore, the particular features, structures, or characteristics may be combined in any suitable manner in one or more aspects.

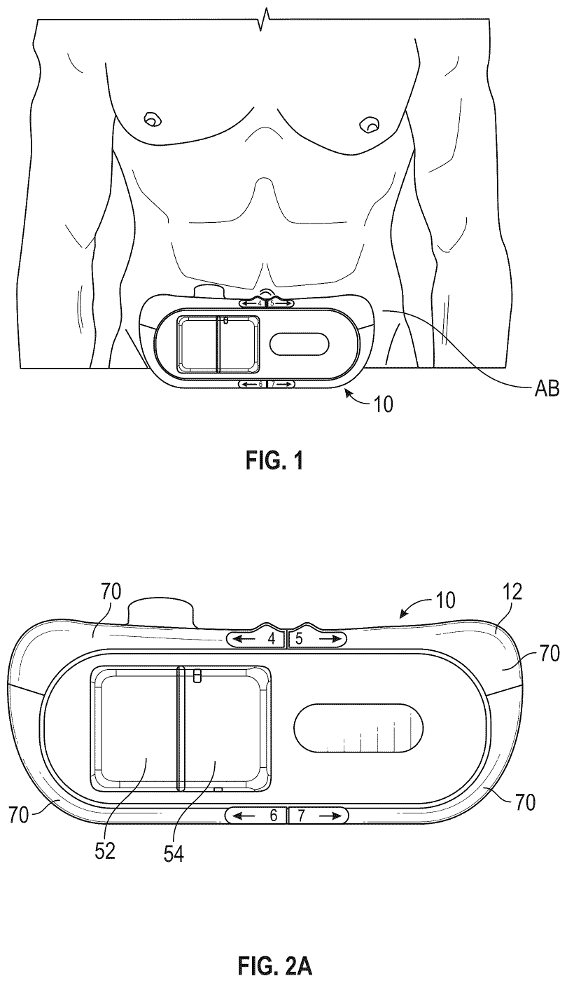

Attention is first drawn to FIG. 1 in which a patch, generally designated 10, is shown adhered to a patient's abdominal region AB, and constituting a part of a diagnostic system, which further includes an in-vivo device (not shown) configured for being introduced into the patient's gastrointestinal system. As can be seen, the patch 10 is fitted just below the navel of the patient and covers a substantial portion of the bottom abdominal region thereof.

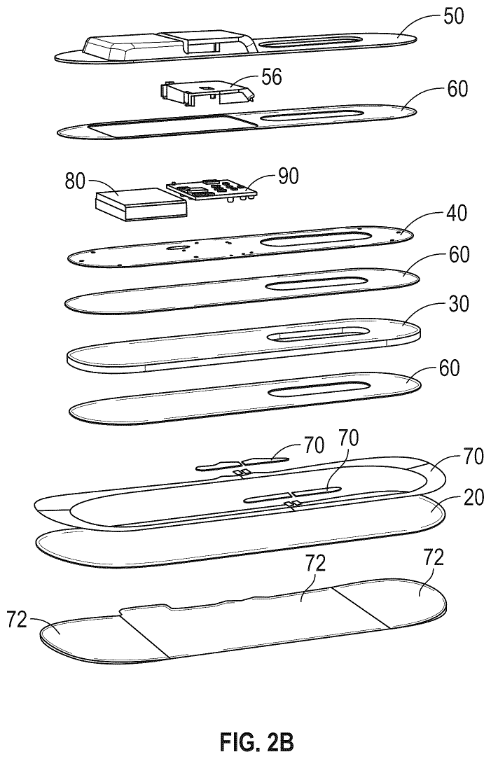

With additional reference being made to FIGS. 2A and 2B, the patch 10 includes a patch body 12 having a plurality of layers including (but not limited to):
an adhesive layer 20 configured for being in direct contact with the patient's body and for fixing the position of the patch with respect to the patient's body;
a spacer layer 30 configured for distancing any electrical components of the patch 10 from the patient's skin;
a communication layer 40 in the form of a printed antenna;
an external cover layer 50;
two intermediate adhesive layers 60; and
a plurality of removable films 70.

The patch 10 further includes a power unit 80 and a processing unit 90 nested within respective inclusions 52 and 54 of the external cover layer 50.

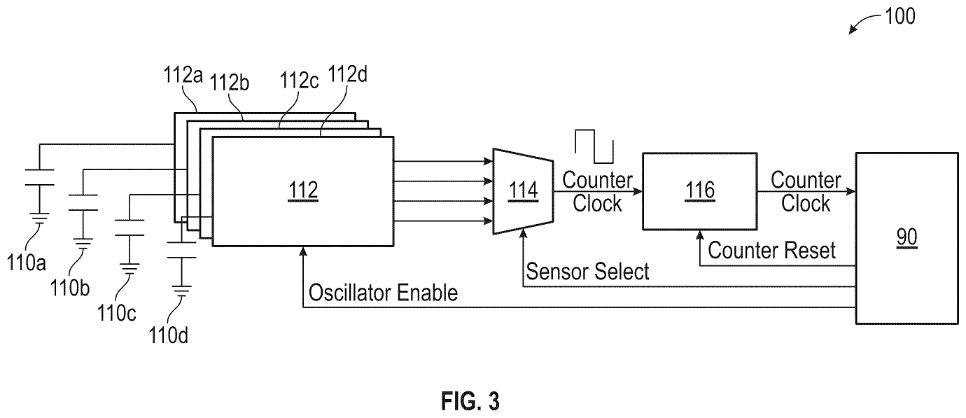

The communication layer 40 includes a sensor arrangement (shown in FIG. 3) which, in conjunction with processing unit 90 form, inter alia, a capacitance detecting arrangement 100 configured for monitoring the electrical capacitance between the patch 10 and the patient's skin. In particular, the capacitance detection arrangement 100 of the present disclosure is sensitive to the distance between the patch 10 and the patient's skin, whereby monitoring capacitance allows alerting the patient and/or health care practitioner regarding full/partial detachment of the patch 10 from the skin. It should be noted that since the patch 10 is configured for being in constant communication with the in-vivo device, full/partial detachment of the patch 10 from the skin may greatly affect communication with the in-vivo device and the patch's 10 ability of receiving/sending signals to and from the in-vivo device respectively.

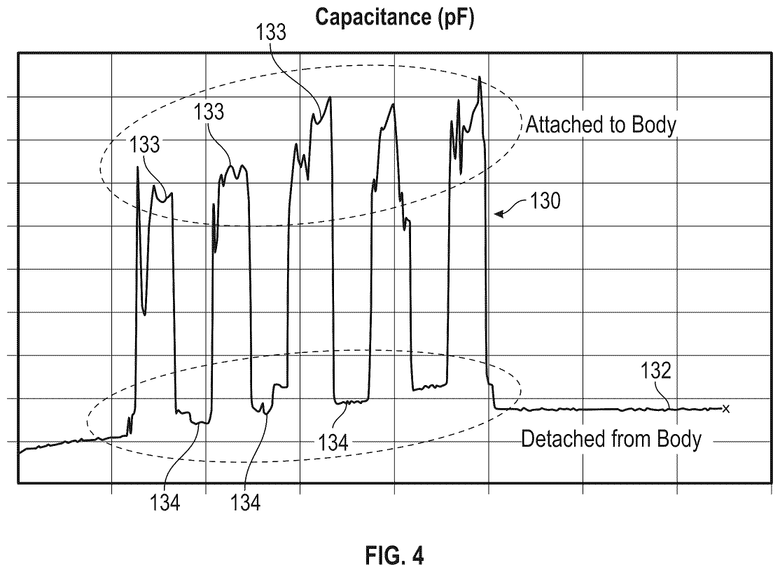

With additional reference being made to FIG. 4, a graph is shown, generally designated 130, demonstrating the capacitance measured by the sensor arrangement when the patch 10 is attached/detached from the patient's skin. The graph 130 is shown where the horizontal axis denotes time (in seconds), and the vertical axis denotes the capacitance (in pico-Farads).

When the patch 10 is completely detached from the patient's body, the sensor arrangement provides a baseline reading 132. The graph 130 represents experimental data yielded when the patch was alternately fitted and removed from the patient's skin. As can be seen, when the patch 10 is properly fitted to the patient's skin, the capacitance spikes up to peaks 133, ranging between 8.8 to 10.3 pF, while, when the patch 10 is detached from the patient's skin, capacitance drops to troughs 134, ranging between 6.2 to 6.7 pF.

This change in capacitance is sufficiently significant to be detectable during operation of the patch 10, whereby the patient or healthcare practitioner may be alerted to a detachment via a variety of possible signals, including (but not limited to): light, vibration, text message, and/or sound, etc.

Reverting to FIG. 3, the implementation of the capacitance detection arrangement 100 is shown including four capacitance sensors 110a to 110d, each connected to respective capacitance sensing oscillators 112a to 112d. The capacitance sensors 110a to 110d may be positioned at different locations along the patch 10, thereby allowing individual monitoring of adhesion of said locations to the patient's skin. In particular, such an arrangement allows alerting the patient not only to the fact that the patch 10 is detached from the body, but also indicate which portion of the patch 10 became detached.

The capacitance sensing oscillators 112a to 112d are coupled to a selector 114, configured for selecting an output signal from the oscillators 112a to 112d in order to sample each of the capacitance sensors periodically and individually.

The arrangement is such that each oscillator 112, when enabled, generates a square wave signal 115 at its output. The frequency of the square wave 115 may vary in a certain range, inverse-proportional to the sensing capacitor 110 value. The oscillator output signal, chosen by the selector 114, is used as input to a counter 116. Before each measurement, the counter 116 is reset and then enabled for a constant time window. At the end of the window, the counter 116 readout is proportional to the oscillator frequency and inverse-proportional to the sensor capacitance. The circuit is calibrated with two known capacitors, so the offset and the slope constants are recorded in Non Volatile Memory (NVM). Using these constants and the counter readout, the CPU 90 calculates the real capacitance measured by the sensors 110a-110d, and can then perform the following:
If the capacitance monitored indicates lower values corresponding to the expected reading of the patch 10 when detached, the CPU 90 may send out a signal to activate the alert mechanism, indicating, to the user, that there is a problem;
If the capacitance monitored indicates high capacitance values corresponding to the expected reading of the patch 10 when properly placed, no action is taken.

In accordance with different variations of the present application, the capacitance sensors 110a-110d of the sensor arrangement 100 can be placed inside the spacing layer 30, externally to the spacing layer (i.e. such that the spacing layer 30 is intermediate between the sensor arrangement 100 and the patient's skin, or even internally to the spacing layer 30.

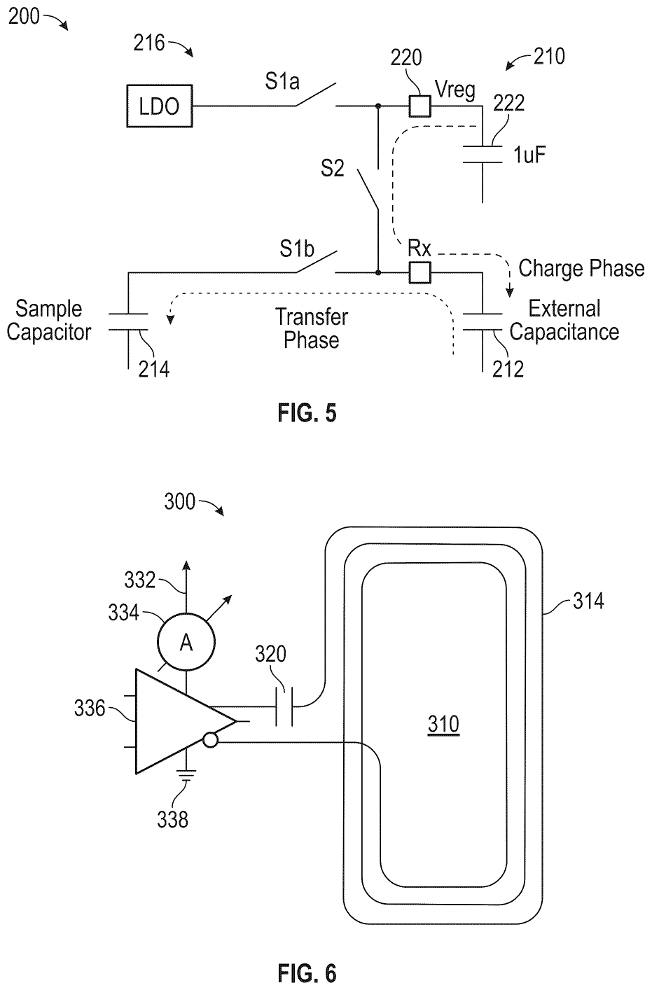

Further attention is drawn to FIG. 5, where another example of a capacitance arrangement is shown, generally designated 200, which is based on self capacitance, which is the capacitance relative to earth ground. In order to measure the self capacitance, charge is transferred between three difference capacitors—an external capacitor 212, an internal sampling capacitor 214, and a Vreg capacitor 222. First, the charge stored on the Vreg capacitor 222 (recommended value of 1 uF) is used to charge the external unknown capacitance 212 via closed switch S2 during the charge phase. Second, the charge from the external capacitance 212 is transferred to an internal sampling capacitor 214 via closed switch S1b. During this transfer phase, when charge is moved from the external capacitor 212 to the sample capacitor 214, the Vreg capacitor 222 is refilled with charge by the low dropout regulator (LDO) 216 via closed switch Sla (with switch S2 opened). These charge and transfer phases are repeated until the voltage on the internal sampling capacitor 214 changes by the desired amount.

Attention is now drawn to FIG. 6, in which another example of an adhesion detection arrangement is shown, generally designated 300, based on an antenna 310, which may be the printed antenna in the communication layer 40 (FIG. 2B). Specifically, the antenna 310 may be used for a downlink channel used for transmitting commands from the patch 10 to the in-vivo device. The antenna 310 is in the form of a coil of several turns 314 located close to the perimeter of the patch 10. The arrangement 300 also includes a capacitor 320, a voltage source connection 332, a current sensor 334, a driver 336, and a ground connection 338. The driver 336 is a high efficiency switching differential amplifier or an equivalent driver. In such embodiments, the amount of the power supply current consumption, as measured by the current sensor 334, is directly proportional to the amount of current though the antenna 310. As described below, the current measured by the current sensor 334 may be used for adhesion detection.

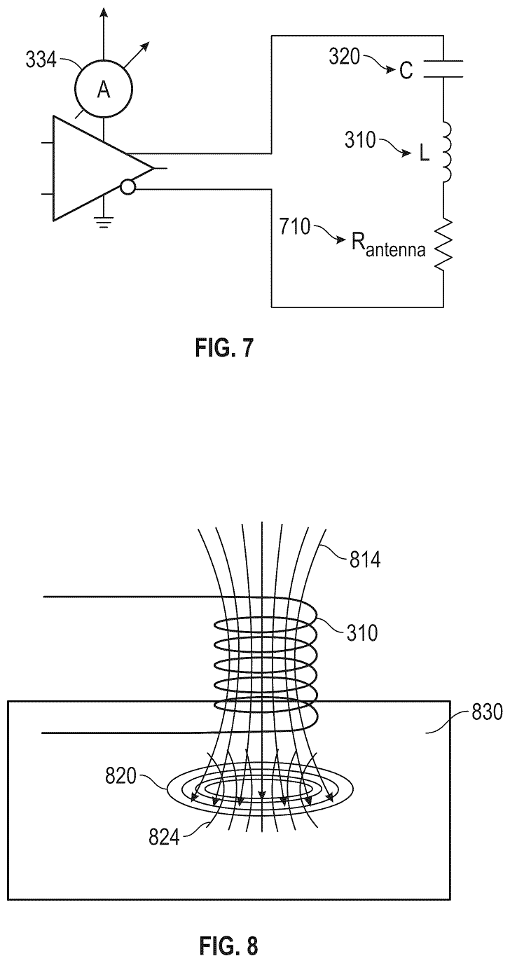

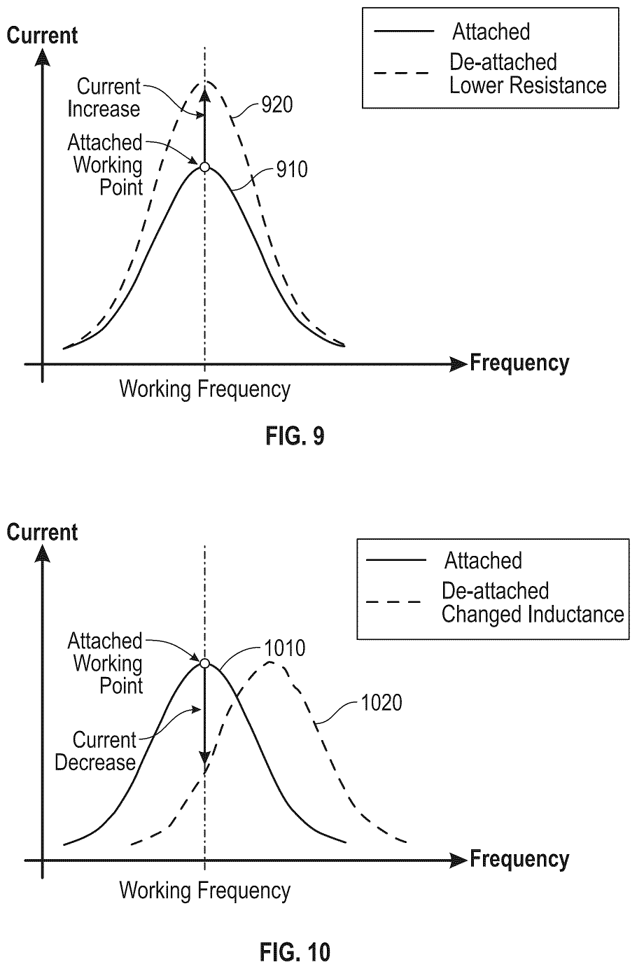

FIG. 7 shows an equivalent circuit for the arrangement 300 of FIG. 6. The capacitor 314 has a capacitance C, and the antenna 310 has an inductance L and an effective resistance Rₐₙₜₑₙₙₐ. The effective resistance reflects the amount of power dissipated by antenna effects in terms of a resistance value. The antenna effects include, for example, radiation resistance, ohmic resistance of metal components, losses from induced currents (e.g., Eddy currents), and/or dielectric losses, among other effects. The capacitor 320 and the antenna 310 form a resonant circuit. The characteristics of the resonant circuit will be described in more detail below in connection with FIGS. 9-11. The values of the inductance L and the effective resistance Rₐₙₜₑₙₙₐ change depending on the immediate environment, which is described in more detail below. For now, it is sufficient to note that, when the patch 10 adheres to a human body, the arrangement 300 and antenna 310 are close to the human body and the antenna has a certain value of the inductance L. When the patch 10 loses adhesion and the arrangement 300 and antenna 310 are farther from the human body, the value of the inductance L changes (either higher or lower) and the effective resistance Rₐₙₜₑₙₙₐ decreases. These effects produce a meaningful change in the current measured by the current sensor 334, such that the measured change in current is usable for adhesion detection. The following describes these effects in more detail.

With respect to the effective resistance Rₐₙₜₑₙₙₐ, the effective resistance Rₐₙₜₑₙₙₐ is illustrated in FIG. 7 as in series with the capacitor 320 and the inductance L of the antenna 310. When the arrangement 300 and antenna 310 are close to the body tissue, power losses attributable to induced eddy currents are present. Eddy currents are described below in connection with FIG. 8. When the arrangement 300 and antenna 310 are detached or are farther from the body tissue, less eddy currents or no Eddy currents are induced. In the latter situation, the effective resistance Rₐₙₜₑₙₙₐ of the antenna 310 decreases due to less power loss from the Eddy current effects. Because the effective resistance Rₐₙₜₑₙₙₐ when the arrangement 300/antenna 310 is detached is less than when the arrangement 300/antenna 310 is attached, the effect of decreased effective resistance is an increase in current. If the decrease in effective resistance is the dominant affect dictating the current, then the current sensed by the current sensor 334 will be greater when the arrangement 300/antenna 310 is detached than the current when the arrangement 300/antenna 310 is attached. Such an increase in the current sensed by the current sensor 334 is usable for detachment detection.

With respect to the inductance L, and with reference also to FIG. 8, when the antenna 310 is positioned close to a human body, the AC current in the coils of the antenna 310 produce a magnetic field 814, which induces eddy currents 820 in the human tissue 830, as shown in FIG. 8, due to the human tissue's conductivity. The eddy currents 820 produce their own magnetic field 824, which interacts with the magnetic field 814 and affects the inductance of the antenna coils. For example, if the inductance has a value L_{attached} when the arrangement 300/antenna 310 are attached to a body and has a value L_{detached} when the arrangement 300/antenna 310 are detached from the body, then L_{attached} would be different from (either greater than or less than) L_{detached}. This is because the magnetic field 824 from the eddy currents 820 is fairly contained, so it has less effect on the arrangement 300/antenna 310 when they are farther from the body. As explained below, the change in inductance changes the current characteristics of the resonant circuit formed by the capacitor 320 and the antenna 310, and when this effect is the dominant effect dictating the current, such change in current is measurable by the current sensor 334 and can be used for adhesion detection.

As mentioned above, and with reference again to FIG. 7, the capacitor 320 and the antenna 310 form a resonant circuit. A resonant circuit has a resonant frequency at which current through the capacitor and the inductor is maximized. If a resonant circuit is operated at a frequency different from the resonant frequency, the current through the capacitor and the inductor would be less.

In accordance with aspects of the present disclosure, the arrangement 300 is operated at a frequency designated the “working frequency,” and the capacitance C of the capacitor 320 is selected so that the working frequency is the resonant frequency for the capacitor 320 and the antenna 310 when the patch 10 is fully attached to a human body. Specifically, when the patch 10 is fully attached to the human body, the antenna 310 has inductance L_{attached}, and the working frequency is the resonant frequency based on capacitance C and inductance L_{attached}. When the working frequency is the resonant frequency, the impedance of the equivalent circuit shown in FIG. 7 is pure resistance with no reactance.

As mentioned above, detaching the antenna from the human body fully or partially will cause the resistive portion of the impedance to decrease and/or cause the inductance L of the antenna 310 to change (either increase or decrease). A decrease in the resistive portion of the impedance has the effect of increasing the current through the resonant circuit. Meanwhile, a change in the inductance L of the antenna 310 from L_{attached} to L_{detached} causes the working frequency to no longer be the resonant frequency of the resonant circuit. Therefore, a change in the inductance L of the antenna 310 has the effect of decreasing the current through the resonant circuit.

These two possible effects of detachment are illustrated in FIGS. 9-11.

FIG. 9 illustrates the effect of detachment resulting in lower effective resistance but no change or negligible change to the resonant frequency. Curve 910 is an example of the current through the resonant circuit at different frequencies when the patch 10 is attached. The curve 920 is an example of the current through the resonant circuit at different frequencies when the patch 10 is detached. Because the example of FIG. 9 results in no change or negligible change to the resonant frequency, the result of detachment and lower effective resistance is an increase in the current through the resonant circuit. In various embodiments, increases in current sensed by the current sensor 334 can be compared to a threshold, such that current increases greater than the threshold can be determined to be a detachment. As mentioned above, assuming that the driver 336 is a high efficiency switching differential amplifier, the power supply current consumption current is directly proportional to the antenna current. The built-in current sensor 334, therefore, is used as the detachment sensor. The measure of the current change depends on the degree of detachment. The maximal current change is caused by full detachment, whereas partial detachment causes a lesser degree of current change.

FIG. 10 illustrates the effect of detachment resulting in lower inductance but no change or negligible change to the effective resistance. Curve 1010 is an example of the current through the resonant circuit at different frequencies when the patch 10 is attached. The curve 1020 is an example of the current through the resonant circuit at different frequencies when the patch 10 is detached. Because the example of FIG. 10 results in no change or negligible change to the effective resistance, the result of detachment and changed inductance is a change in the resonant frequency, such that the working frequency is no longer the resonant frequency. Accordingly, the current through the resonant circuit at the working frequency decreases based on the change in resonant frequency. In various embodiments, decreases in current sensed by the current sensor 334 can be compared to a threshold, such that current decreases greater than the threshold can be determined to be a detachment. As mentioned above, assuming that the driver 336 is a high efficiency switching differential amplifier, the power supply current consumption current is directly proportional to the antenna current. The built-in current sensor 334, therefore, is used as the detachment sensor. The measure of the current change depends on the degree of detachment. The maximal current change is caused by full detachment, whereas partial detachment causes a lesser degree of current change. The increase in resonant frequency shown in FIG. 10 is merely an example. Decreases in resonant frequency are contemplated to be within the scope of the present disclosure.

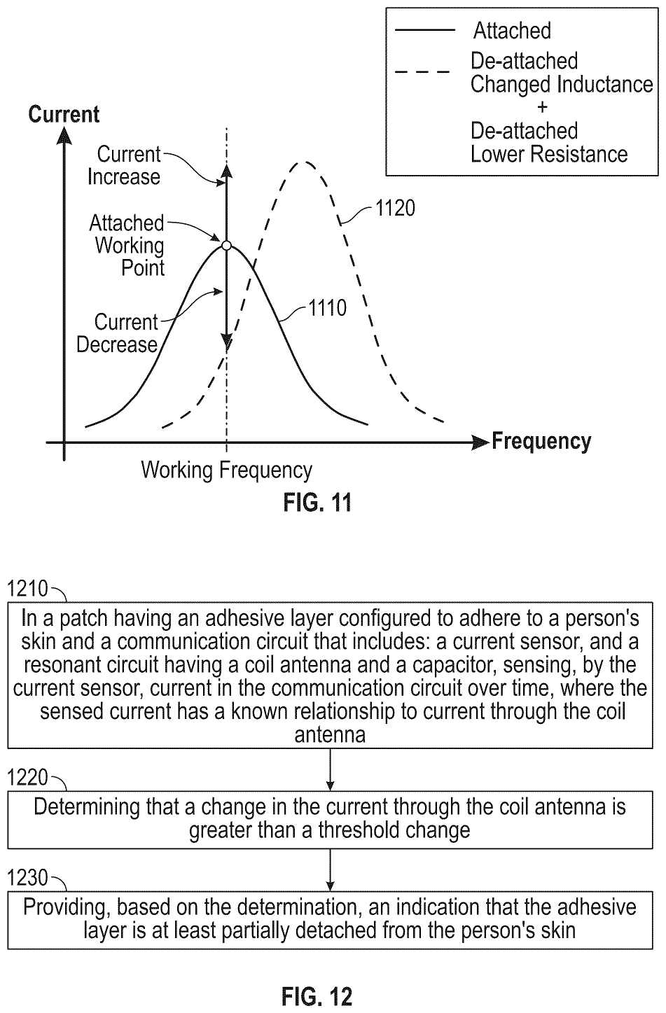

FIG. 11 illustrates the effect of detachment resulting in lower effective resistance and a change in inductance, and accordingly, a change in the resonant frequency. Curve 1110 is an example of the current through the resonant circuit at different frequencies when the patch 10 is attached. The curve 1120 is an example of the current through the resonant circuit at different frequencies when the patch 10 is detached. Because the example of FIG. 11 results in change in the resonant frequency and decrease in the effective resistance, the resulting current is the net result of increase in the current due to lower effective resistance and decrease in the current due to a change in resonant frequency. In various embodiments, the change in current sensed by the current sensor 334 can be compared to a threshold, such that current changes (either increase or decrease) greater than the threshold can be determined to be a detachment. As mentioned above, assuming that the driver 336 is a high efficiency switching differential amplifier, the power supply current consumption current is directly proportional to the antenna current. The built-in current sensor 334, therefore, is used as the detachment sensor. The measure of the current change depends on the degree of detachment. The maximal current change is caused by full detachment, whereas partial detachment causes a lesser degree of current change. The increase in resonant frequency shown in FIG. 11 is merely an example. Decreases in resonant frequency are contemplated to be within the scope of the present disclosure.

FIG. 12 is a flow diagram of an operation for adhesion detection for the detector of FIG. 6. At block 1210, the operation is in a patch having an adhesive layer configured to adhere to a person's skin and a communication circuit that includes: a current sensor, and a resonant circuit having a coil antenna and a capacitor. The operation involves sensing, by the current sensor, current in the communication circuit over time, where the sensed current has a known relationship to current through the coil antenna. The known relationship may be, for example, directly proportionality, inverse proportionality, or another known relationship.

At block 1220, the operation involves, determining that a change in the current through the coil antenna is greater than a threshold change. The change in the current may be an increase in current or may be a decrease in current. The change in current may be cause by decreased resistance in the resonant circuit and/or decreased inductance of the coil antenna.

At block 1230, the operation involves, providing, based on the determination, an indication that the adhesive layer is at least partially detached from the person's skin. The indication may include visual, audio, and/or tactile indications, among others.

Those skilled in the art to which this invention pertains will readily appreciate that numerous changes, variations, and modifications can be made without departing from the scope of the invention, mutatis mutandis.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

The systems, devices, and/or servers described herein may utilize one or more processors to receive various information and transform the received information to generate an output. The processors may include any type of computing device, computational circuit, or any type of controller or processing circuit capable of executing a series of instructions that are stored in a memory. The processor may include multiple processors and/or multicore central processing units (CPUs) and may include any type of device, such as a microprocessor, graphics processing unit (GPU), digital signal processor, microcontroller, programmable logic device (PLD), field programmable gate array (FPGA), or the like. The processor may also include a memory to store data and/or instructions that, when executed by the one or more processors, causes the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PLI, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

1. A patch comprising:
an adhesive layer configured to adhere to skin of a person;
a communication circuit comprising a current sensor and a resonant circuit comprising a coil antenna and a capacitor, wherein the current sensor is configured to sense current in the communication circuit over time, wherein the sensed current has a known relationship to current through the coil antenna; and
at least one of a circuit or a processor configured to at least perform:
determining that a change in the current through the coil antenna is greater than a threshold change, and
providing, based on the determination, an indication that the adhesive layer is at least partially detached from the skin of the person.

2. The patch of claim 1, wherein the communication circuit operates at a working frequency, wherein the working frequency is configured to be a resonant frequency of the resonant circuit when the adhesive layer is fully adhered to the skin of the person.

3. The patch of claim 2, wherein the working frequency is not the resonant frequency of the resonant circuit when the adhesive layer is at least partially detached from the skin of the person.

4. The patch of claim 1, wherein the determining that the change in the current through the coil antenna is greater than a threshold change comprises determining that an increase in the current through the coil antenna is greater than the threshold change.

5. The patch of claim 1, wherein the determining that the change in the current through the coil antenna is greater than a threshold change comprises determining that a decrease in the current through the coil antenna is greater than the threshold change.

6. The patch of claim 1, wherein the communication circuit is configured to transmit signals to an in-vivo device within the person, wherein the change in the current through the coil antenna is sensed during a transmission by the communication circuit.

7. A method in a patch comprising an adhesive layer configured to adhere to skin of a person and a communication circuit comprising: a current sensor, and a resonant circuit comprising a coil antenna and a capacitor, the method comprising:
sensing, by the current sensor, current in the communication circuit over time, wherein the sensed current has a known relationship to current through the coil antenna;
determining that a change in the current through the coil antenna is greater than a threshold change; and
providing, based on the determination, an indication that the adhesive layer is at least partially detached from the skin of the person.

8. The method of claim 7, further comprising:
operating the communication circuit at a working frequency,
wherein the working frequency is configured to be a resonant frequency of the resonant circuit when the adhesive layer is fully adhered to the skin of the person.

9. The method of claim 8, wherein the working frequency is not the resonant frequency of the resonant circuit when the adhesive layer is at least partially detached from the skin of the person.

10. The method of claim 7, wherein the determining that the change in the current through the coil antenna is greater than a threshold change comprises determining that an increase in the current through the coil antenna is greater than the threshold change.

11. The method of claim 7, wherein the determining that the change in the current through the coil antenna is greater than a threshold change comprises determining that a decrease in the current through the coil antenna is greater than the threshold change.

12. The method of claim 7, further comprising:
transmitting, by the communication circuit, signals to an in-vivo device within the person,
wherein the change in the current through the coil antenna is sensed during a transmission by the communication circuit.

13. A non-transitory processor readable medium storing instructions which, when executed by at least one processor of a patch comprising an adhesive layer configured to adhere to skin of a person and a communication circuit comprising: a current sensor, and a resonant circuit comprising a coil antenna and a capacitor, causes the patch to at least perform:
accessing data representing sensed current, sensed by the current sensor, of current in the communication circuit over time, wherein the sensed current has a known relationship to current through the coil antenna;
determining that a change in the current through the coil antenna is greater than a threshold change; and
providing, based on the determination, an indication that the adhesive layer is at least partially detached from the skin of the person.

14. The non-transitory processor readable medium of claim 13, wherein the instructions, when executed by the at least one processor, further cause the patch to at least perform:
operating the communication circuit at a working frequency,
wherein the working frequency is configured to be a resonant frequency of the resonant circuit when the adhesive layer is fully adhered to the skin of the person.

15. The non-transitory processor readable medium of claim 14, wherein the working frequency is not the resonant frequency of the resonant circuit when the adhesive layer is at least partially detached from the skin of the person.

16. The non-transitory processor readable medium of claim 13, wherein the determining that the change in the current through the coil antenna is greater than a threshold change comprises determining that an increase in the current through the coil antenna is greater than the threshold change.

17. The non-transitory processor readable medium of claim 13, wherein the determining that the change in the current through the coil antenna is greater than a threshold change comprises determining that a decrease in the current through the coil antenna is greater than the threshold change.

18. The non-transitory processor readable medium of claim 13, wherein the instructions, when executed by the at least one processor, when further cause the patch at least to perform:
transmitting, by the communication circuit, signals to an in-vivo device within the person,
wherein the change in the current through the coil antenna is sensed during a transmission by the communication circuit.
